(19) United States
(12) Patent Application Publication
(10) Pub. No.: US 2008/0051379 A1
(43) Pub. Date: Feb. 28, 2008

(54) Compositions and Methods for the Treatment of Peripheral B-Cell Neoplasms

(75) Inventors: Adam Lerner, Newton, MA (US); Sanjay Tiwari, Buchholz (DE)

Correspondence Address: RONALD I. EISENSTEIN, 100 SUMMER STREET, NIXON PEABODY LLP, BOSTON, MA, 02110, US

(73) Assignee: Trustees of Boston University, Boston, MA (US)

(21) Appl. No.: 11/792,172

(22) PCT Filed: Dec. 1, 2005
(86) PCT No.: PCT/US05/43613

§ 371 Date: Nov. 6, 2007

Related U.S. Application Data
(60) Provisional application No. 60/632,207, filed on Dec. 1, 2004.

(51) Int. Cl. A61K 31/56; A61P 35/02

(52) U.S. Cl. 514/171

(57) ABSTRACT

The present invention is directed to the use of a PDE4 inhibitor and a glucocorticoid to treat peripheral B-cell neoplasms. In particular, the present invention provides a method of treating individuals (e.g. patients) diagnosed with peripheral B-cell leukemias by administering pharmaceutical compositions comprising Type 4 cyclic adenosine monophosphate phosphodiesterase inhibitors and a glucocorticoid. Preferably, the combination of the PDE4 inhibitor and the glucocorticoid has a synergistic effect on apoptosis such that the level of apoptosis induced is greater than the level that would be expected by simply adding a PDE4 inhibitor to a glucocorticoid.

### CROSS-REFERENCE TO RELATED APPLICATION

This application claims the benefit under 35 U.S.C. 119(e) of U.S. Provisional Patent Application Ser. No. 60/632,207, filed Dec. 1, 2004, the contents of which are herein incorporated by reference in their entirety.

### GOVERNMENT SUPPORT

This invention was made with Government Support under Contract No. CA79838 was awarded by the National Institutes of Health. The Government has certain rights in the invention.

### FIELD OF THE INVENTION

The present invention is directed to the treatment of patients with chronic, peripheral B-cell neoplasms, including B-cell chronic lymphocytic leukemia (CLL), with pharmaceutical compositions comprising a glucocorticoid and a Type 4 cyclic adenosine monophosphate phosphodiesterase (PDE4) inhibitor.

### BACKGROUND OF THE INVENTION

Leukemias are malignant neoplasms of hematopoietic tissues. These neoplasms are categorized into two predominant forms: chronic and acute. Acute leukemias (ALLs) are characterized by undifferentiated, rapidly growing cell populations. ALLs are more common among children. Chronic leukemias (CLLs) usually present a more mature morphology and affects adults more than children.

However, in addition to the acute and chronic categorization, neoplasms are also categorized based upon the cells giving rise to such disorder into precursor or peripheral. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, diffuse large B-cell lymphoma and Burkitt lymphoma. Notwithstanding these classifications, however, the pathological impairment of normal hematopoiesis is the hallmark of all leukemias.

Chronic lymphocytic leukemia (CLL) is a neoplasm characterized by the clonal expansion of small lymphocytes, which accumulate in the marrow, lymph nodes, blood, spleen, liver, and sometime other organs. The CLL cell is the neoplastic counterpart of an immunologically immature, incompetent lymphocyte. In over 95 percent of cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype.

CLL is the most prevalent leukemia afflicting adults in modern countries, accounting for 30 percent of all leukemias. The American Cancer Society estimates that, in 2004, there will be about 8,190 new cases of chronic lymphocytic leukemia (CLL) in the US. About 4,800 people in the US will die of CLL during 2004. Chronic lymphocytic leukemia affects only adults. The average age of patients is about 70; it is rarely seen in people under the age of 40.

Most patients are diagnosed following a routine physical examination or a blood count. The earliest and most frequent symptoms are fatigue and malaise. Later symptoms include lymphadenopathy and splenomegaly. Anemia and thrombocytopenia are found in approximately 15 percent of patients.

The general goal of leukemia therapy is to arrest the proliferation of abnormal morphologies and restore “normal” hematopoiesis in the bone marrow. Treatment regimens include chemotherapy. Unfortunately, chemotherapy is not always successful. Indeed, while CLL patients may have initial clinical responses to alkylating agents such as chlorambucil or adenosine analogs such as fludarabine, many ultimately become refractory to therapy. Consequently, there is a pressing need for the identification of novel approaches to this disease.

Glucorticoids have an apoptotic effect on different cells, including lymphocytic leukemia cells, and have been used for example in combination with other cancer therapeutics to treat ALL, a precursor neoplasm (Kato et al., Blood 82:2304-9 (1993); Ogawa et al., Blood 92:2484-94 (1998)). Glucocorticoids have also been used in combination with other therapies to treat B-CLL. (Zilio et al., Blood 100:4974 (2002); Tsukada et al., Blood 100:3166 (2002); Tsukada et al., Blood 98: 40b-41b (2001)). Specific subsets of normal and malignant B and T lineage lymphoid cells are unique in their sensitivity to the induction of apoptosis by agents that increase intracellular levels of the second messenger cAMP¹⁻³. The same subsets of lymphoid cells are unusually sensitive to the induction of apoptosis by glucocorticoids⁴⁻⁶. Several groups have identified similarities in the signaling pathways activated by these two stimuli in such cell types. Early studies demonstrated that certain genes were up-regulated in lymphoid cells by both glucocorticoids and cAMP analogs⁷. Subsequent studies by McConkey and colleagues demonstrated that in CCRF-CEM cells, a human lymphoid cell line derived from a patient with T-acute lymphocytic leukemia, loss of glucocorticoid receptor (GR) led to loss of sensitivity to cAMP-induced apoptosis⁸. Glucocorticoid and protein kinase A (PKA) signaling pathways have also been shown to synergize in inducing apoptosis in glucocorticoid-resistant CCRF-CEM cells⁹⁻¹¹. Interestingly, cAMP-mediated potentiation of glucocorticoid-induced apoptosis has been reported to be independent of cAMP response element (CRE)-associated transcriptional activation¹². Most recently, the catalytic subunit of PKA was found to associate with GR¹³.

The mechanism by which glucocorticoids induce lymphoid apoptosis remains unclear. GR signaling both positively and negatively regulates transcription. While positive regulation of gene transcription is mediated through palindromic GRE elements, several mechanisms for negative regulation of gene transcription by the GR have been described including negative GREs, composite elements and tethering¹⁴⁻¹⁶. Surprisingly, most of the clinically beneficial activities of glucocorticoids, such as inhibition of lymphoid proliferation and inflammatory cytokine secretion, appear to be mediated by a tethering mechanism, in which GR suppresses NFκB or AP1-mediated transcription in a manner independent of the ability of the GR to bind to DNA itself¹⁷.

Studies examining glucocorticoid and cAMP-mediated apoptosis have typically utilized leukemic cell lines as the experimental model. However, primary leukemic cells differ in important ways from such immortalized cell lines, most strikingly in that primary cells fail to proliferate to any significant degree in tissue culture.

Cyclic AMP is catabolized within cells to 5′-AMP by 3′:5′ cAMP phosphodiesterases (PDE), a diverse group of enzymes which have proven to be the target of successful pharmaceutical agents for neurologic, cardiovascular and inflammatory disorders (21, 22). Despite this large array of cyclic nucleotide PDEs, only a subset of these enzymes have been reported in human lymphoid cells. Among them, the most commonly reported enzymes in human T cells are types 1, 3 and 4. Calcium-calmodulin dependent type 1 PDE activity has been detected in phytohemagglutinin-stimulated but not resting peripheral blood lymphocytes. One isoform from this family, PDE1B1, has been detected in acute lymphocytic leukemia cells; inhibition of this enzyme was reported to induce apoptosis. PDE1 enzymes, which can catalyze the degradation of both cAMP and cGMP, are specifically inhibited by vinpocetine (IC50=21 mMol/L). Type 4 cAMP phosphodiesterase (PDE4) is the principal enzyme responsible for the catabolism of cAMP to 5′-AMP in lymphoid cells¹⁸⁻²⁰. Two groups have reported both type 3 and type 4 PDE in human T lymphocytes; lectin-mediated proliferation was completely suppressed only by treating cells with specific inhibitors of both classes of enzymes.

As a result of differential expression and subcellular localization, PDE4 isoforms vary in their signal transduction properties. It has been previously demonstrated that rolipram, a prototypic PDE4-specific inhibitor, induces apoptosis in B-CLL cells but not peripheral blood T cells, by a mitochondrial pathway and in a PKA-dependent manner (26-29; U.S. Pat. No. 6,399,649). However, there remains a population of B-CLL patients which have leukemic cells relatively resistant to rolipram. Weintraub et al., Blood 98: 284b (2001).

PDE4 inhibitors have also been used in combination with other agents such as fludarabine (Welsh et al., Blood 96:758a (2000); see also Siegmund et al., Leukemia 15:1564-71 (2001); Moon et al., Blood 101:4122-30 (2003)).

Accordingly, there is a need for improved methods and compositions to treat peripheral B-cell neoplasm, and in particular B-CLL.

### SUMMARY OF THE INVENTION

We have now discovered that a combination of a PDE4 inhibitor and a glucocorticoid surprisingly induces high levels of apoptosis in peripheral B-cell neoplasms such as primary B-CLL cells.

Accordingly, the present invention provides a method of treating individuals (e.g. patients) with peripheral B-cell leukemias by administering pharmaceutical compositions comprising Type 4 cyclic adenosine monophosphate phosphodiesterase inhibitors and a glucocorticoid. Preferably, the combination of the PDE4 inhibitor and the glucocorticoid has a synergistic effect on apoptosis such that the level of apoptosis induced is greater than the level that would be expected by simply adding a PDE4 inhibitor to a glucocorticoid.

One embodiment of the present invention provides a method comprising: a) selecting a patient having symptoms of peripheral B-cell leukemia; and b) co-administering to said patient a therapeutically effective amount of i) an inhibitor that specifically inhibits Type 4 cyclic adenosine monophosphate phosphodiesterases (i.e. a PDE4 inhibitor), and ii) a glucocorticoid.

The peripheral B-cell leukemia includes a B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, B cell non-Hodgkin's lymphoma and Waldenstrom's macroglobulineamia. Preferably, the peripheral B-cell neoplasm is a primary B-CLL, B-CLL, multiple myeloma, B cell non-Hodgkin's lymphoma, mantle cell lymphoma and Waldenstrom's macroglobulinemia.

In one preferred embodiment of the present invention, the Type 4 cyclic adenosine monophosphate phosphodiesterase inhibitor is rolipram or RO20-1724. Preferred glucocorticoids include hydrocortisone and dexamethosone.

The present invention is not limited by the method of administration. In one embodiment, the administration is enteral administration. In another embodiment, said enteral administration is oral administration. In still another embodiment, said administration is parenteral administration. In these embodiments, said parenteral administration can be for example topical administration or by a transdermal patch. In another embodiment, said parenteral administration is subcutaneous administration. While in still another embodiment, said parenteral administration utilizes an aerosol.

The present invention is not limited by the nature of the patient. In one embodiment, said patient is a naive patient (e.g., has not undergone prior treatment for CLL), while in other embodiments said patient is unresponsive or refractory to standard chemotherapy (e.g., alkylating agents). In still another embodiment, said patient is immunocompromised. In one embodiment, said patient is over fifty years of age.

The present invention is also not limited by the method of determining response to treatment. In one embodiment, said symptoms comprise lymphadenopathy and splenomegaly. In a yet another embodiment, said symptoms comprise the histology of a lymph node that is consistent with CLL.

The PDE4 inhibitor and the glucocorticoid can be administered simultaneously/concurrently or sequentially. When administered sequentially, this can be, for example, administered one day to 1 month after each other; it is preferred that they are administered within one week of each other, more preferably within three days, still more preferably within two days, even more preferably within one day, and most preferably within 12 hours of each other.

However, one can administer either one of the compounds multiple times before the co-administration.

Any inhibitor which specifically inhibits PDE4 can be used in the present invention. Preferred inhibitors include, but are not limited to, rolipram (4-(3-cyclopentyloxy-4-methoxyphenyl)-2-pyrrolidone (CAS [61413-54-5]); 4-(3-Butoxy-4-methoxybenzyl)-2-imidazolidinone, referred to herein as RO20-1724 (Schwabe et al., 1972; Sheppard et al., 1972); Piclamilast (Ashton et al., J. Med. Chem. 27: 1696-1703 (1994); a 9-benzyladenine derivative nominated NCS-613 (INSERM); D-4418 from Chiroscience and Schering-Plough; mesopram (Merz et al., J. Med. Chem. 41:4733-43 (1998)); a benzodiazepine PDE4 inhibitor identified as CI-1018 (PD-168787; Parke-Davis/Warner-Lambert); a benzodioxole derivative Kyowa Hakko disclosed in WO 99/16766; PMNPQ (6-(4-pyridylmethyl)-8-(3-nitrophenyl)quinoline; see Correa-Sales et al., J. Pharmacol. Exp. Therap. 263:11046-9 (1992); Robichaud et al., Br. J. Pharmacol. 135:113-8 (2002)); V-11294A from Napp (Landells, L. J. et al. Eur Resp J [Annu Cong Eur Resp Soc (September 19-23, Geneva) 1998] 1998, 12(Suppl. 28): Abst P2393); roflumilast (CAS reference No. 162401-32-3); a pthalazinone (WO 99/47505) from Byk-Gulden; a compound identified as T-440 (Tanabe Seiyaku; Fujii, K. et al. J Pharmacol Exp Ther, 1998, 284(1): 162); cis 4-cyano-4-(3-cyclopentyloxy-4-methoxyphenyl)cyclohexan-1-carboxylic acid; 2-carbomethoxy-4-cyano-4-(3-cyclo-propylmethoxy-4-difluoromethoxyphenyl)cyclohexan-1-one; cis-[4-cyano-4-(3-cyclopropylmethoxy-4-difluoromethoxyphenyl)cyclohexan-1-ol]; cilomalast (cis-4-cyano-4-[3-(cyclopentyloxy-)-4-methoxyphenyl]cyclohexane-1-carboxylic acid), as well as other compounds set out in U.S. Pat. No. 5,552,438; L-826,141 [4-{2-(3,4-Bisdifluromethoxyphenyl)-2-{4-(1,1,1,3,3,3-hexafluoro-2-hydroxypropan-2-yl)-phenyl]-ethyl}-3-methylpyridine-1-1-oxide], J. Pharmacol. Exp. Ther., Aug. 1, 2004; 310(2): 752-760; AWD 12-343 (Hofgen N, Egerland U, Poppe H, et al.; paper presented at: 27th National Medicinal Chemistry Symposium, Kansas City Mo., Jun. 16, 2000; poster B-19); 7-benzylamino-6-chloro-2-piperazino-pteridine (DC-TA-4C; Laurent et al., WO 97/15561); N-(3,5-Dichloro-pyrid-4-yl)-[1-(4-fluorobenzyl)-5-hydroxy-indole-3-yl]-glyoxylic acid amide (AWD-12-281 from elbion (Hofgen, N. et al. 15th EFMC Int Symp Med Chem (September 6-10, Edinburgh) 1998, Abst P.98; CAS reference No. 247584020-9); K-34 from Kyowa Hakko; arofylline, under development by Almirall-Prodesfarma; VM554/UM565 from Vernalis; and salts, esters, pro-drugs, and analogues thereof.

Any glucocorticoid can be used in the present invention. Preferred glucocorticoiods include, but are not limited to, betamethasone, budesonide, cortisone, cortisone acetate, dexamethasone, hydrocortisone, methylprednisolone, prednisolone, prednisone, triamcinolone, methylprednisolone, triamcinolone, beclomethasone, fludrocortisone acetate, deoxycorticosterone acetate (DOCA), and aldosterone.

In one preferred embodiment, the present invention provides the use of further combination therapy, such as a PDE4 inhibitor and a glucocorticoid, in combination with other drugs, including but not limited to cytotoxic drugs.

### BRIEF DESCRIPTION OF THE DRAWINGS

FIGS. 1A-D show the effects of the cyclic nucleotide phosphodiesterase inhibitors rolipram, RO20-1724 and cilostamide on hydrocortisone and dexamethasone-induced apoptosis in B-CLL. In FIG. 1A, leukemic cells from 11 B-CLL patients were assessed for apoptosis by Hoechst 3342 analysis after 48 hrs culture in media alone (No treatment), 10 μM rolipram (Roli), 1 μM hydrocortisone (HC) or a combination of the two drugs (Roli+HC). The clinical characteristics of the B-CLL patients are summarized in Table 1. In FIGS. 1B and 1C, leukemic cells from five patients with B-CLL were cultured in media alone or with a dose titration of hydrocortisone (1B: solid line) or dexamethasone (1C: solid line), followed by assessment for apoptosis by Hoechst 33342 FACS analysis at 48 hours. Using the same experimental conditions, the effect of addition of 10 μM rolipram (thick dashed line) to the glucocorticoids was also assessed. In FIG. 1D, the apoptotic effects of the PDE4 inhibitor RO20-1724 (RO20) (10 μM) or the PDE3 inhibitor cilostamide (Cilost) (10 μM) were assessed on B-CLL cells when combined with 1 μM hydrocortisone. Data represent the mean±SEM of triplicate samples.

FIG. 2 shows a comparison of the apoptotic effect of rolipram and hydrocortisone treatment on B-CLL, normal peripheral blood T, and CD3+ CD4+ Sezary cells. Magnetic bead purified peripheral blood T cells or leukemic cells from patients with B-CLL or Sezary syndrome were cultured in media alone or with a dose titration of hydrocortisone (solid line), followed by assessment for apoptosis by Hoechst 33342 FACS analysis at 48 hours. Using the same experimental conditions, the effect of addition of 10 μM rolipram (thick dashed line) or 10 μM rolipram and 40 μM forskolin (dotted line) was also assessed. The SEM of triplicate samples is shown; where not visible, the SEM was less than 1.0%.

FIGS. 3A-B show that hydrocortisone-induced transactivation of GRE elements is augmented by PDE4 inhibition and adenylyl cyclase stimulation in B-CLL and CCRF-CEM cells, respectively. B-CLL cells (FIG. 3A) or CCRF-CEM cells (FIG. 3B) were transiently transfected with a luciferase construct in which expression is regulated by GRE elements, followed by culture in 10 μM rolipram (R), 40 μM forskolin (Fsk), 1 μM hydrocortisone (HC), 1 mM Rp-8Br-cAMPS (Rp-8Br) or combinations of these agents. 12 hours after addition of the drugs, the relative luminescence of the samples was determined. The data shown are the mean of 8 experiments for B-CLL and two experiments for CCRF-CEM cells. The single asterisk denote a significant difference by paired student's t test between HC alone and either R/HC (FIG. 3A) or F/HC (FIG. 3B) (p<0.02). The double asterisk denotes a significant difference between Rp-8Br-cAMPS/HC and HC alone (p<0.02).

FIG. 4 shows that glucocorticoid-induced apoptosis in B-CLL is abrogated by the PKA antagonist Rp-8Br-cAMPS. B-CLL cells from six patients were cultured for 48 hours in either media alone (Control), 1 μM hydrocortisone (HC), or 1 μM hydrocortisone in combination with 1 mM Rp-8Br-cAMPS (HC+Rp-8Br). Cells were assessed for apoptosis by Hoechst 33342 flow cytometry. Data represent the mean±SEM of triplicate samples.

FIGS. 5A-C show that the diterpene adenylyl cyclase stimulant forskolin synergizes with glucocorticoids in inducing apoptosis in dexamethasone-sensitive and resistant CCRF-CEM cells. In FIG. 5A, the polyclonal parental cell line (CCRF-CEM), the single cell-derived glucocorticoid-resistant subclone (CEM-R8) and the single cell-derived glucocorticoid-sensitive subclone (CEM-S2) were cultured for 72 hours in the presence of vehicle (ETOH) or dexamethasone at the concentrations indicated and cell viability was assessed by the MTS assay as described in Methods. In FIG. 5B, the glucocorticoid-resistant subclone (CEM-R8) and the glucocorticoid-sensitive subclone (CEM-S2) were cultured for 72 hours in the presence of hydrocortisone (HC) (10 μM HC for the CEM-R8 cells and 1 μM HC for the CEM-S2 cells), 10 μM forskolin (Fsk), 10 μM rolipram (Rol), or combinations of these agents as indicated, and cell viability was assessed by the MTS assay as described in Methods. FIG. 5C: The glucocorticoid-resistant subclone (CEM-R8) and the glucocorticoid-sensitive subclone (CEM-S2) were cultured for 72 hours in the presence of varying amounts of dibutyryl cAMP (db-cAMP) in the absence (−HC) or presence (+HC) of 1 μM hydrocortisone as indicated, and cell viability was assessed by the MTS assay as described in Methods. The data shown were normalized to the vehicle control. Results represent the mean±SD of triplicate determinations. Similar results were obtained in a total of three experiments performed.

FIG. 6 shows that PDE4 inhibitors raise cAMP levels in B-CLL but not CCRF-CEM cells, while forskolin raises cAMP levels in CCRF-CEM. Leukemic cells from six B-CLL patients or CCRF-CEM cells were incubated for 30 minutes with media alone (CT), 10 μM rolipram (Roli), 10 μM forskolin (Fsk), 1 μM hydrocortisone (HC), or the same concentration of hydrocortisone combined with rolipram (Roli/HC) or forskolin (Fsk/HC). Lysates of the cells were then assayed for cAMP using a RIA. The cAMP level obtained from each treated B-CLL sample was normalized to that observed in the untreated leukemic cells. The CCRF-CEM data shown are representative of three experiments performed.

FIG. 7 shows that B-CLL and CCRF-CEM cells differ in PDE4 isoform expression following treatment with rolipram and forskolin. B-CLL and CCRF-CEM cells were incubated for 18 hours with media alone, 10 μM rolipram, 10 μM forskolin (Fsk), 1 μM hydrocortisone (HC), or the same concentration of rolipram combined with 1 μM hydrocortisone (Roli+HC) or 10 μM forskolin (Roli+Fsk). Lysates of the cells were then assessed for expression of PDE4A, PDE4B or PDE4D by Western analysis. Equal loading of samples was verified by immunoblotting for tubulin.

### DETAILED DESCRIPTION OF THE INVENTION

The present invention relates to the surprising discovery that the combination of two agents, a PDE4 inhibitor and a glucocorticoid, induces apoptosis in peripheral B-cell leukemias such as primary B-CLL cells and multiple myeloma cells in a synergistic manner, i.e. to a greater extent than would be expected by simply adding a PDE4 inhibitor to a glucocorticoid.

Accordingly, the present invention provides a method of treating patients with peripheral B-cell leukemias such as chronic lymphocytic leukemia (B-CLL) by co-administering a therapeutically effective amount of a Type 4 cyclic adenosine monophosphate phosphodiesterase inhibitor (i.e. a PDE4 inhibitor) and a glucocorticoid. Preferably, the combination of the PDE4 inhibitor and the glucocorticoid has a synergistic effect on apoptosis such that the level of apoptosis induced is greater than the level that would be expected by simply adding a PDE4 inhibitor such as rolipram and RO20-1724 to a glucocorticoid, such as hydrocortisone and dexamethosone.

The peripheral B-cell leukemias to be treated by the present methods include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, B cell non-Hodgkin's lymphoma and Waldenstrom's macroglobulineamia. Preferred peripheral B-cell leukemias include primary B-CLL, B-CLL, multiple myeloma, B-cell non-Hodgkin's lymphoma, mantle cell lymphoma and Waldenstrom's macroglobulinemia. A preferred peripheral B-cell neoplasm is B-cell CLL.

Any specific inhibitor of PDE4 can be used in the compositions and methods of the present invention. In one embodiment, the PDE4 inhibitor is rolipram [4-(3-cyclopentyloxy-4-methoxphenyl)-2-pyrrolidone], CAS [61413-54-5] (IC50=1 mMol/L) or the structurally related compound RO20-1724, also known as XX5 ((4-(3-Butoxy-4-methoxybenzyl)-2-imidazolidinone)(IC50=2 mMol/L). U. Schwabe et al., “4-(3-cyclopentyloxy-4-methoxyphenyl)-2-pyrrolidone (ZK 62711): a potent inhibitor of adenosine cyclic 3′,5′-monophosphate phosphodiesterases in homogenates and tissue slices from rat brain,” Molecular Pharmacology 12:900 (1976). H. Sheppard et al., “Structure-activity relationships for inhibitors of phosphodiesterase from erythrocytes and other tissues,” Adv Cyclic Nucl Res 1:103 (1972).

Other inhibitors include but are not limited to piclamilast (Ashton et al., J. Med. Chem. 27: 1696-1703 (1994); a 9-benzyladenine derivative nominated NCS-613 (INSERM); D-4418 from Chiroscience and Schering-Plough; mesopram (Merz et al., J. Med. Chem. 41:4733-43 (1998)); a benzodiazepine PDE4 inhibitor identified as CI-1018 (PD-168787; Parke-Davis/Warner-Lambert); a benzodioxole derivative Kyowa Hakko disclosed in WO 99/16766; PMNPQ (6-(4-pyridylmethyl)-8-(3-nitrophenyl)quinoline; see Correa-Sales et al., J. Pharmacol. Exp. Therap. 263:11046-9 (1992); Robichaud et al., Br. J. Pharmacol. 135:113-8 (2002)); V-11294A from Napp (Landells, L. J. et al. Eur Resp J [Annu Cong Eur Resp Soc (September 19-23, Geneva) 1998] 1998, 12(Suppl. 28): Abst P2393); roflumilast (CAS reference No. 162401-32-3); a pthalazinone (WO 99/47505) from Byk-Gulden; a compound identified as T-440 (Tanabe Seiyaku; Fujii, K. et al. J Pharmacol Exp Ther, 1998, 284(1): 162); cis 4-cyano-4-(3-cyclopentyloxy-4-met-hoxyphenyl)cyclohexan-1-carboxylic acid; 2-carbomethoxy-4-cyano-4-(3-cyclo-propylmethoxy-4-difluoromethoxyphenyl)cyclohexan-1-one; cis-[4-cyano-4-(3-cyclopropylmethoxy-4-difluoromethoxyphenyl)cyclohexan-1-ol]; cilomalast (cis-4-cyano-4-[3-(cyclopentyloxy-)-4-methoxyphenyl]cyclohexane-1-carboxylic acid), as well as other compounds set out in U.S. Pat. No. 5,552,438; L-826,141 [4-{2-(3,4-Bisdifluromethoxyphenyl)-2-{4-(1,1,1,3,3,3-hexafluoro-2-hydroxypropan-2-yl)-phenyl]-ethyl}-3-methylpyridine-1-oxide], J. Pharmacol. Exp. Ther., Aug. 1, 2004; 310(2): 752-760; AWD 12-343 (Hofgen N, Egerland U, Poppe H, et al.; paper presented at: 27th National Medicinal Chemistry Symposium, Kansas City Mo., Jun. 16, 2000; poster B-19); 7-benzylamino-6-chloro-2-piperazino-pteridine (DC-TA-4C; Laurent et al., WO 97/15561); AWD-12-281 from elbion (Hofgen, N. et al. 15th EFMC Int Symp Med Chem (September 6-10, Edinburgh) 1998, Abst P.98; CAS reference No. 247584020-9); K-34 from Kyowa Hakko; arofylline, under development by Almirall-Prodesfarma; VM554/UM565 from Vernalis; IC485, under development by ICOS; and salts, esters, pro-drugs, and analogues thereof. Other specific PDE4 inhibitors which can be used in the methods of the present invention are described in published U.S. Patent Application Nos. 20030013754 (Martins et al.), 20040152754 (Martins et al.), 20040067954 (Eggenweiler et al.), 20020028842 (Lauener et al.), and 20030220352 (Lauener et al.).

Rolipram, RO 20-1724, roflumilast and cilomalast are preferred. Rolipram is more preferred. Other specific PDE4 inhibitors which can be used in the methods of the present invention are described in published U.S. Patent Application Nos. 20030013754 (Martins et al.), 20040152754 (Martins et al.), 20040067954 (Eggenweiler et al.), 20020028842 (Lauener et al.), and 20030220352 (Lauener et al.). In one embodiment the PDE4 inhibitor is an inhibitor of the PDE4B isoform, more preferably the PDE4B2 isoform.

Glucocorticoids are steroid hormones characterized by their ability to bind to the cortisol receptor. Glucocorticoids are well known to those of skill in the art and any glucocorticoid known to those of skill in the art with these characteristics can be used in the compositions and methods of the present invention. Preferred glucocorticoiods include, but are not limited to, betamethasone, budesonide, cortisone, cortisone acetate, dexamethasone, hydrocortisone, methylprednisolone, prednisolone, prednisone, triamcinolone, methylprednisolone, triamcinolone, beclomethasone, fludrocortisone acetate, deoxycorticosterone acetate (DOCA), and aldosterone.

The clinical use of glucocorticoids is described for example in detail in the Physicians' Desk Reference, 56^{th} Ed. (2002) Publisher Edward R. Barnhart, New Jersey (“PDR”).

In an alternative embodiment, any glucocorticoid except hydrocortisone or dexamethasone is used in combination with any PDE4 inhibitor.

In an alternative embodiment, any glucocorticoid is used in combination with any PDE4 inhibitor except rolipram or RO20-1724.

In yet another alternative embodiment, any glucocorticoid except hydrocortisone or dexamethasone is used in combination with any PDE4 inhibitor except rolipram or RO20-1724.

The compositions of the present invention are useful for the treatment of any peripheral B-cell leukemia, including, but not limited to, B-cell chronic lymphocytic leukemia (B-CLL). In general, the methods and compositions of the invention may be used to treat or alleviate the symptoms of a patient suffering from peripheral B-cell leukemia, such as B-CLL.

Preferably, the compositions and methods of the invention induce apoptosis in the peripheral B-cell leukemic cells. More preferably, apoptosis is induced in at least 20% of the peripheral B-cell leukemic cells, preferably in at least 40% of the cells, more preferably at least 60%, yet more preferably 80%, even more preferably, at least 90% of the cells compared to a similar population of untreated cells. Most preferably, 100% of the peripheral B-cell leukemic cells have apoptosis induced. Most preferably, at least 95% of the peripheral B-cell leukemic cells have apoptosis induced.

Treatment regimens of patients with peripheral B-cell leukemic cells, such as CLL, including administration of the combination therapies of the present invention, can be tailored for different stages and types of the disease. For example, chronic lymphocytic leukemia does not usually form a tumor. It generally involves all of the bone marrow in the body and, in many cases, has spread to other organs such as the liver, spleen, and lymph nodes when it is found. Therefore the prognosis of the leukemia depends on other information, such as its type or subtype, cellular features determined by lab tests, and results of imaging studies.

In one embodiment, the treatment regimen comprises a PDE4 inhibitor and glucocorticoid in combination with an alkylating agent. Alkylating agents and appropriate dosing is known to those of skill in the art. Common alkylating agents useful in the present invention include chlorambucil, adenosine analogs such as fludarabine, carboplatin and paclitaxel.

In one embodiment, apoptosis can be enhanced by providing sufficient basal adenylyl cyclase activity to drive cAMP accumulation in a subcellular compartment. Accordingly, the PDE4 inhibitor and glucocorticoid of the present invention can be co-administered with any agent which increases cAMP accumulation in a subcellular compartment, including by increasing basal adenylyl cyclase activity. Such agents are well known in the art.

There are 2 different systems for staging neoplasms such as peripheral B-cell leukemic cells, for example, CLL. The Rai classification is used more often in the United States, whereas the Binet system is used more widely in Europe. The Rai stages can be separated into low-, intermediate-, and high-risk categories. Stage 0 is considered low risk, stages I and II are considered intermediate risk, and stages III and IV are considered high risk. The Rai classification recognizes 5 stages. Rai Stage 0: Lymphocytosis is present (the blood lymphocyte count is too high, usually defined as over 10,000 lymphocytes per cubic millimeter (mm3) of blood. Some doctors will diagnose, for instance, CLL if the count is over 5,000/mm3 and the cells all have the same chemical pattern on special testing). The lymph nodes, spleen, and liver are not enlarged and the red blood cell and platelet counts are near normal. Rai Stage I: Lymphocytosis plus enlarged lymph nodes. The spleen and liver are not enlarged and the red blood cell and platelet counts are near normal. Rai Stage II: Lymphocytosis plus enlarged liver or spleen, with or without enlarged lymph nodes. The red blood cell and platelet counts are near normal. Rai Stage III: Lymphocytosis plus anemia (too few red blood cells), with or without enlarged lymph nodes, spleen, or liver. Platelet counts are near normal. Rai Stage IV: Lymphocytosis plus thrombocytopenia (too few blood platelets), with or without anemia, enlarged lymph nodes, spleen, or liver.

In the Binet staging system, a neoplasm such as CLL is classified according to the number of affected lymphoid tissue groups (neck lymph nodes, groin lymph nodes, underarm lymph nodes, spleen, and liver) and the presence of anemia (too few red blood cells) or thrombocytopenia (too few blood platelets). Binet Stage A: Fewer than 3 areas of lymphoid tissue are enlarged, with no anemia or thrombocytopenia. Binet Stage B: 3 or more areas of lymphoid tissue are enlarged, with no anemia or thrombocytopenia. Binet Stage C: Anemia and/or thrombocytopenia are present.

Prognostic factors for a peripheral B-cell leukemic cell such as CLL are also considered in developing treatment regimens. In addition to a patient's stage, there are other factors that help predict his or her outlook for survival. These factors are sometimes used in addition to staging information when deciding possible treatment options. Factors that tend to be associated with shorter survival time are called adverse prognostic factors. Those that predict longer survival are favorable prognostic factors.

Adverse prognostic factors for such leukemias include the following. Diffuse pattern of bone marrow involvement (more widespread replacement of normal marrow by leukemia). Abnormal chromosome changes—except for partial deletions of chromosome 13, which are good. High blood levels of certain substances, such as beta-2-microglobulin. Increased proportion of large or atypical lymphocytes in blood samples.

The combination therapies of the present invention can be used in conjunction with any other therapeutic regimens used to treat peripheral B-cell lymphomas, including but not limited to chemotherapy such as chlorambucil and fludarabine, monoclonal antibodies such as Alemtuzumab and Rituximab, radiation therapy for certain patients, splenectomy, and/or stem cell transplantation.

The pharmaceutical combination or each agent individually can be administered by any means known in the art. Such modes include oral, rectal, nasal, topical (including buccal and sublingual), or parenteral (including subcutaneous, intramuscular, intravenous, and intradermal) administration, including sustained release formulations.

For ease to the patient, oral administration is preferred. However, typically oral administration requires a higher dose than an intravenous administration. Thus, administration route will depend upon the situation: the skilled artisan must determine which form of administration is best in a particular case, balancing dose needed versus the number of times per month administration is necessary.

In administering the compounds one can use the normal dose of each compound individually.

The first component of the combination therapy described is a PDE4 inhibitor. The PDE4 inhibitor may be administered in any manner found appropriate by a clinician, such as described on a product label, or in the clinical literature, or in the Physicians' Desk Reference, 56^{th} Ed. (2002) Publisher Edward R. Barnhart, New Jersey (“PDR”). For example, when the PDE4 inhibitor is rolipram, the dosage is 0.5-50 mg/kg; preferably 1-10 mg/kg, but any dosage within the general range appropriate to the patient can be used.

The second component of the combination therapy described is a glucocorticoid. The choice of a particular glucocorticoid to treat an individual is influenced by many factors, including the stage of the leukemia, the age and general health of the patient, and issues of multidrug resistance. The glucocorticoid may be administered in any manner found appropriate by a clinician, such as those described for individual glucocorticoids such as described on a product label, or in the clinical literature or in the PDR. For example, when the glucocorticoid is hydrocortisone, the dose is preferably 5-500 mg/day, more preferably 20-240 mg/day but any dosage within the general range appropriate to the patient can be used.

“Pharmaceutically acceptable” as used herein means that the salts and derivatives of the PDE4 inhibitors and glucocorticoids having the same general pharmacological properties as the free acid form from which they are derived and are acceptable from a toxicity viewpoint.

As with the use of other pharmaceutical compositions, the individual patient will be monitored in a manner deemed appropriate by the treating physician.

The pharmaceutical compositions of this invention which are found in combination may be in the dosage form of solid, semi-solid, or liquid such as, e.g. suspension, aerosols, or the like. Preferably the compositions are administered in unit dosage forms suitable for single administration of precise dosage amounts. The compositions may also include, depending on the formulation desired, pharmaceutically-acceptable, nontoxic carriers or diluents, which are defined as vehicles commonly used to formulate pharmaceutical compositions for animal or human administration. Compositions may be provided as sustained release or timed release formulations. The carrier or diluent may include any sustained release material known in the art, such as glyceryl monostearate or glyceryl distearate, alone or mixed with a wax. Microencapsulation may also be used. The timed release formulation can provide a combination of immediate and pulsed release throughout the day. The diluent is selected so as not to affect the biological activity of the combination. Examples of such diluents are distilled water, physiological saline, Ringer's solution, dextrose solution, and Hank's solution. In addition, the pharmaceutical composition of formulation may also include other carriers, adjuvants, emulsifiers, or nontoxic, nontherapeutic, nonimmunogenic stabilizers and the like. Effective amounts of such diluent or carrier will be those amounts which are effective to obtain a pharmaceutically acceptable formulation in terms of solubility of components, or biological activity, and the like.

In therapeutic applications, the dosages and administration schedule of the agents used in accordance with the invention vary depending on the agent, the age, weight, and clinical condition of the recipient patient, and the experience and judgment of the clinician or practitioner administering the therapy, among other factors affecting the selected dosage. Generally, the dose and administration scheduled should be sufficient to result in slowing, and preferably regressing, the growth of the tumor(s) and also preferably causing complete regression of the cancer. In some cases, regression can be monitored by a decrease in blood levels of tumor specific markers. An effective amount of a pharmaceutical agent is that which provides an objectively identifiable improvement as noted by the clinician or other qualified observer.

The agents in combination, or separately, are delivered at periodic intervals that can range from several times a day to once per month. As noted above, the agents are administered until the desired therapeutic outcome has been obtained. Additionally, in order to avoid side-effects not all components of the combination need to be delivered at each administration. For example, if the combination is administered twice a week the individual components can be administered only once a week (every second treatment).

This invention further includes pharmaceutical combinations comprising a PDE4 inhibitor and a glucocorticoid, as provided above and kits for the treatment of patients with peripheral B-cell neoplasms, comprising a vial of the PDE4 inhibitor and a vial of the glucocorticoid, at the doses provided above. Most preferably, the kit contains instructions describing their use in combination.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications, and publications cited herein are incorporated herein by reference in their entirety.

As used herein and throughout, the following words or terms have the following meaning: leukemia is synonymous with neoplasm; CLL is meant to indicate chronic lymphocytic leukemia; and PDE4 is meant to indicate a type 4 cyclic adenosine monophosphate phosphodiesterase.

### EXAMPLES

Materials and Methods

Reagents

The following reagents were obtained from commercial sources: cilostamide and rolipram (Calbiochem, San Diego, Calif.); forskolin, 1,9 dideoxyforskolin, PMS (phenazine methosulfate) (Sigma Chemical Co., St. Louis, Mo.); Hoechst 33342 and DiOC₆(3) (3,3′-dihexyloxacarbocyanine iodide) (Molecular Probes, Eugene, Oreg.); MTS and St-Ht31 AKAP inhibitor peptide (Promega, Madison, Wis.); RO20-1724 (Biomol, Plymouth Meeting, Pa.), (Rₚ)-8-Br-cAMPS (Biolog, Bremen, Germany). IC242 was a kind gift from Dr. Sharon Wolda, ICOS (Bothell, Wash.)²⁶.

Patient Selection

Blood samples were obtained by IRB-approved consent from flow cytometry-confirmed B-CLL patients that were either untreated or for whom at least one month had elapsed since chemotherapy. Patients with active infections or other serious medical conditions were not included in this study.

Cell Purification and Culture

CCRF-CEM cells were obtained from ATCC²⁷. Leukemic or normal mononuclear cells were obtained by centrifugation over Histopaque 1077 (Sigma Chemical Company, St. Louis, Mo.). For purification of T cells, whole mononuclear cells from normal subjects were incubated with magnetic beads coated with appropriate antibodies, then positively purified using a magnet (Miltenyi). Cells were cultured in RPMI 1640 media (Biowhittaker, Walkersville, Md.) supplemented with 10% fetal calf serum, 50 μMol/L 2-mercaptoethanol, 2 mMol/L L-glutamine, 10 mM Hepes pH 7.4, 100 μg/ml penicillin, and 100 U/ml streptomycin (Sigma Chemical Company, St. Louis, Mo.). For isolation of glucocorticoid resistant clones, parental CCRF-CEM cells were treated with 1 μM dexamethasone for 10 days, and surviving cells diluted to ≦one cell/well in a 96-well flat-bottom tissue culture plate, grown for three weeks in media supplemented with 20% fetal calf serum and 1% insulin-transferrin-selenium (GIBCO), and then transferred to regular growth medium. A glucocorticoid-resistant subclone (CEM-R8) was completely resistant to dexamethasone-induced apoptosis up to at least 10 μM dexamethasone. For isolation of glucocorticoid-sensitive clones, the same procedure was used, except that treatment with dexamethasone was omitted. A glucocorticoid-sensitive subclone (CEM-S2) was inhibited in its survival by dexamethasone with an IC50=0.007 μM.

Apoptosis and Cell Survival Assays

Hoechst 33342 and DiOC₆(3) apoptosis assays were performed as previously described^{22,28}. For CCRF-CEM cell survival assays, cells were plated at a density of 3×10⁴/well in 96-well flat-bottom plates in the presence of test reagents or vehicle in 0.1 mL media. Following incubation for 72 hrs, 20 μl of a 20:1 MTS (2 mg/mL)/PMS (0.92 mg/ml) solution was added to each well, and the plates incubated for 2 hrs at 37°. The absorbance (O.D.) of the formazan product was determined at 492 nm using a plate reader (Titertek Multiscan Plus, Labsystems). Percent cell viability was calculated as follows: (O.D. control sample−O.D. blank)/(O.D. test sample−O.D. blank)×100. All assays were performed in triplicate.

Transfection Technique

An MMTV GRE luciferase construct, originally created by Dr. R. Evans, was a gift from Dr. Remco Spanjaard (Dept. of Otolaryngology, Boston Medical Center)²⁹. 100 million B CLL cells in 200 μl of PBS were added to 500 μl of Nucleofector Solution D containing 30 μg of endotoxin-free (Qiagen) MMTV luciferase constructs. 100 μl was used per transfection using a Nucleofector electroporation instrument (Amaxa Biosystems, Koeln, Germany) using program U16. Pilot experiments with a GFP expression vector demonstrated 20% transfection efficiency and 50% cell survival at 18 hours. Up to six such aliquots of transfected cells were diluted in media and pooled, prior to distribution of 8 million cells/well in 48 well plates. Two hours after transfection, drugs, cytokines or vehicle alone were added to the transfected cells. 14 hours after transfection, samples were processed using a luciferase assay kit (Promega). Triplicate samples were analyzed with an MGM Instruments Optocomp I luminometer (Hamden, Conn.). Analysis of cell viability (trypan blue exclusion) and total protein (Pierce) verified that equivalent numbers of viable B-CLL cells were analyzed. 100 million CCRF-CEM cells were similarly transfected, cultured and analyzed except that Nucleofector program O17 was utilized. Pilot experiments with a GFP expression vector demonstrated 50% transfection efficiency and viability. Data were analyzed using a two-sided paired t test for means.

cAMP Assay

500,000 B-CLL or CCRF-CEM cells were incubated for 30 minutes in one mL media alone or with the addition of drugs. Cells were centrifuged and lysed in 80% ethanol. After vortexing and incubation on ice for 10 minutes, cellular debris was removed by re-centrifugation. The supernatant was dried in a Speedivac and the sample brought up in 250 μL of sample buffer as provided by the makers of a cAMP RIA kit (Amersham). 100 μL of this sample was used for each cAMP assay. The RIA kit was used according to the manufacturer's instructions.

Western Analysis

30 million B-CLL cells or 10 million CCRF-CEM cells were incubated for 18 hours in media alone or with the addition of drugs as indicated in the text. Western analysis was then carried out as previously described³⁰. The PDE4A (66C12H), PDE4B (96G7A) and PDE4D (61D10E) antibodies were kind gifts from Dr. Sharon Wolda (ICOS, Bothell, Wash.) and have been previously described^{30 31}.

Statistical Analysis

Data are reported as the means±SE. Comparisons between multiple groups were performed using single factor ANOVA and secondary comparisons were performed using Duncan's test.

Results

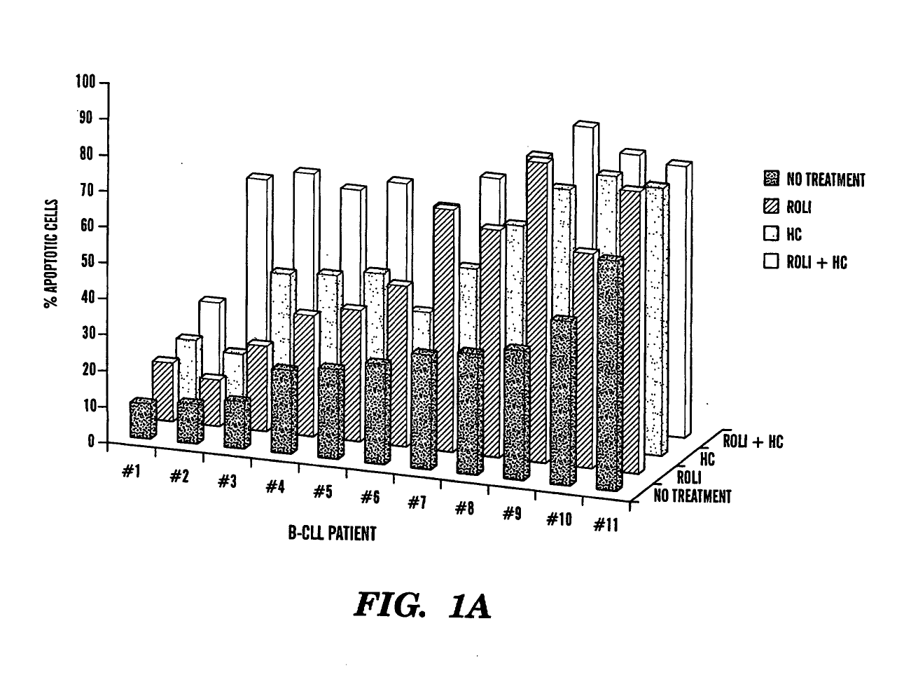

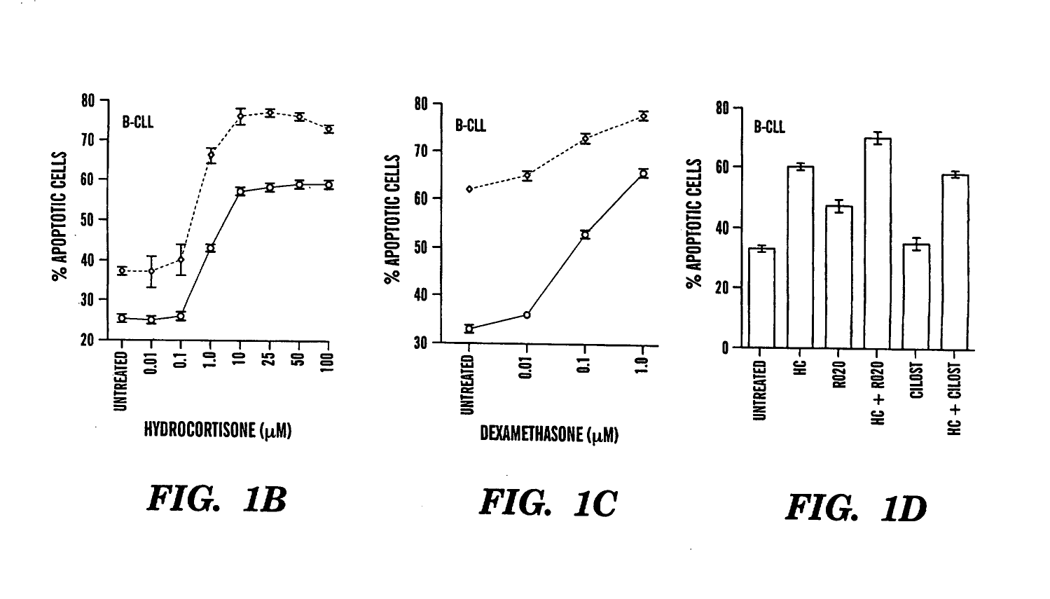

PDE4 inhibitors synergize with glucocorticoids to induce apoptosis in B-CLL but not T cells. To examine the effect of PDE4 inhibition on glucocorticoid-induced apoptosis in B-CLL, leukemic cells were cultured for 48 hours with media alone, the PDE4-specific inhibitor rolipram, the glucocorticoid hydrocortisone, or the two drugs in combination, followed by assessment for apoptosis. A steep dose-response curve for hydrocortisone-induced apoptosis was observed between 0.1 and 10 μM, with little additional effect of hydrocortisone concentrations above 10 μM (FIG. 1B). Subsequently, the effect of combining a glucocorticoid with a PDE4 inhibitor was tested at 1 μM hydrocortisone.

Patient to patient variability was observed both for basal and drug-induced apoptotic rates. However, for all of the eleven leukemic cell samples examined, treatment with a combination of rolipram and hydrocortisone induced a higher percentage of apoptotic B-CLL cells than either agent alone. In patients with high basal levels of apoptosis (Patients #7-11) combined treatment with the two agents induced >65% apoptosis, but this was less than additive of the apoptosis induced by each agent alone (Table 1 and FIG. 1A). In contrast, in five of the six patients with less than 68% apoptosis (Patients #2-6) following combined therapy, treatment with both agents induced a supra-additive or “synergistic” effect (Table 1 and FIG. 1A). It is likely that the lack of synergy observed in the leukemic cell samples with high levels of apoptosis following combined treatment reflects the plateau at 75-85% maximal apoptosis we have observed in B-CLL cells, regardless of the apoptotic stimulus examined, using these in vitro apoptosis assays.

To verify that addition of a PDE4 inhibitor augmented killing of B-CLL cells even when maximally effective doses of glucocorticoids were utilized, rolipram was added to leukemic cell samples treated with hydrocortisone concentrations as high as 100 μM. In five patients so analyzed, addition of 10 μM rolipram augmented the induction of apoptosis by 40±18% relative to the increment in apoptosis induced with 100 μM hydrocortisone alone (FIG. 1B). At lower hydrocortisone concentrations that more closely approximate those achieved following clinical administration of glucocorticoids, the supra-additive effect observed by combining the two agents was again evident. Parallel experiments utilizing DiOC₆(3) to examine mitochondrial depolarization in B-CLL cells treated with glucocorticoids and rolipram confirmed the results obtained above using the Hoechst 33342 apoptosis assay (data not shown).

To generalize these results, we next examined the effects of combined therapy with other drugs within these two classes of compounds. When rolipram was added to the glucocorticoid dexamethasone, the combined treatment once again augmented B-CLL apoptosis beyond the levels observed with either agent alone (FIG. 1C). As with rolipram, the PDE4 inhibitor RO20-1724 (10 μM) augmented hydrocortisone-induced apoptosis in leukemic cells from the five B-CLL patients tested (FIG. 1D). In contrast, despite prior documented expression of PDE3 in B-CLL cells, inhibition of this PDE with the selective inhibitor cilostamide (10 μM), neither induced apoptosis by itself, above that of control, nor augmented hydrocortisone-induced apoptosis in leukemic cells from the same five B-CLL patients (FIG. 1D)³⁰.

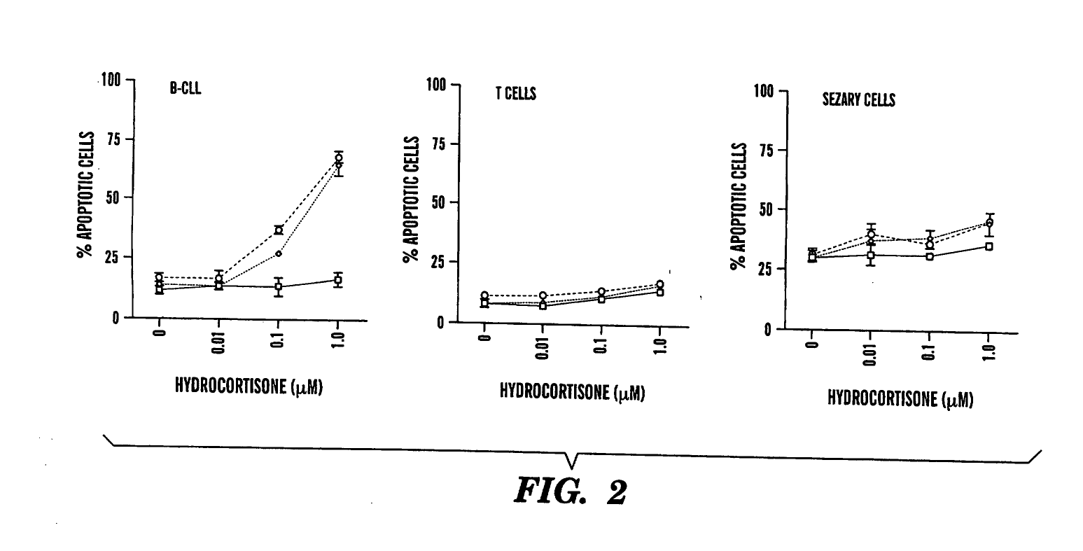

If intermittent therapy of B-CLL patients with regimens that include PDE4 inhibitors and glucocorticoids is to be of clinical benefit, it would be preferable that such therapy does not induce apoptosis in normal T cells, as drug regimens that induce T cell apoptosis are associated with an increased risk of opportunistic infections. As previously published, treatment with 10 μM rolipram failed to induce apoptosis in peripheral blood T cells (FIG. 2)²¹. Further, in contrast to B-CLL cells, rolipram treatment also failed to augment the apoptotic effect of 1 μM hydrocortisone on T cells (FIG. 2). While 10 μM rolipram or 1 μM hydrocortisone used separately had little effect on leukemic cells derived from a patient with Sezary syndrome, a CD3+ CD4+ leukemia, a modest supra-additive effect was seen upon combining the two drugs (FIG. 2). In prior studies of B-CLL, we found that PDE4 inhibitors induce apoptosis and activate PKA and the Rap1 GDP exchange factor EPAC in the absence of additional adenylyl cyclase stimulation²¹⁻²³. Forskolin, a direct activator of adenylyl cyclase, when added at 40 μM, augmented rolipram-induced apoptosis in B-CLL to a minor degree, but had little or no further effect on the combination of rolipram and hydrocortisone in B-CLL cells, peripheral blood T cells or Sezary cells (FIG. 2).

PDE4 Inhibitors Augment Glucocorticoid-Mediated GRE Transactivation in B-CLL

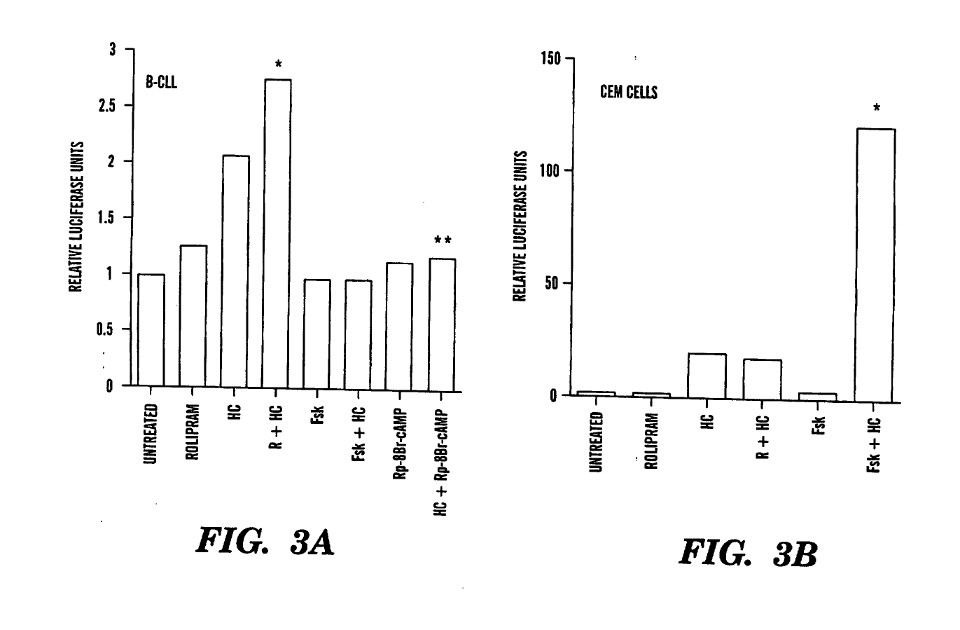

The experiments described above demonstrate that PDE4 inhibitors can synergize with glucocorticoids in inducing apoptosis. It has been reported that PDE4 inhibitors can increase GR-mediated signaling³². To test such a hypothesis, we examined the effects of rolipram on hydrocortisone-induced transactivation of GRE luciferase constructs transiently transfected into B-CLL cells. In 5 patients tested, the addition of 10 μM rolipram significantly augmented hydrocortisone-induced transactivation by an average of 33% (FIG. 3: p<0.02). Although this increase in transactivation is clearly modest, the transfection efficiency of the B-CLL cells was low (10-20% viable transfected cells) which would be expected to limit the augmentation observed. As deletion of the GR has been demonstrated to inhibit cAMP-induced apoptosis in CCRF-CEM cells, we also examined the effect of rolipram itself on GRE transactivation⁸. Although we consistently observed minor augmentation (10%) of GRE transactivation by rolipram (7/8 experiments), the augmentation was not statistically significant (p<0.23). Thus, these data do not support the hypothesis that PDE4 inhibitors induce apoptosis in vitro in B-CLL cells by augmenting basal GRE transactivation through the GR.

Inhibition of PKA Blocks Both Glucocorticoid-Mediated Apoptosis and GRE Transactivation in B-CLL

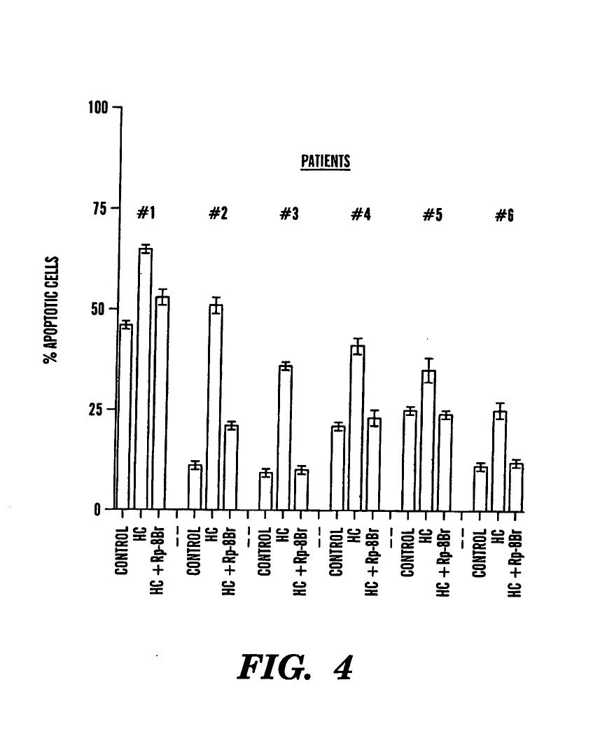

Given the evidence that PDE4 inhibitors, previously shown to induce PKA-mediated signaling in B-CLL cells, can synergize with glucocorticoids to induce apoptosis in B-CLL, we asked whether conversely PKA was required for glucocorticoid-mediated apoptosis. Our prior studies of PKA inhibitors had demonstrated that treatment of B-CLL cells with Rp-8Br-cAMPS (1 mM), an enantiomeric cAMP-binding site competitive antagonist, blocked rolipram-induced CREB phosphorylation and significantly reduced both basal and rolipram-induced B-CLL apoptosis²³. Remarkably, we found that co-treatment of B-CLL cells with 1 mM Rp-8Br-cAMPS inhibited hydrocortisone-induced apoptosis by 86±14% in the six B-CLL patients tested (FIG. 4). Treatment with 1 mM Rp-8Br-cAMPS also reduced hydrocortisone-induced transactivation of GRE luciferase constructs by 83% in 8 B-CLL samples tested (FIG. 3; p<0.02). Preincubation of the B-CLL cells with 10 μM St-Ht31, a membrane-permeable peptide that inhibits the binding of PKA to AKAPs also significantly reduced hydrocortisone-induced transactivation by an average of 33% in three patients tested (P<0.006)³³. These studies suggest that PKA activity is required for both glucocorticoid-mediated GRE transactivation and B-CLL apoptosis.

Adenylyl Cyclase Activation but Not PDE4 Inhibition Augments Glucocorticoid-Induced Apoptosis and GRE Activation in CCRF-CEM Cells

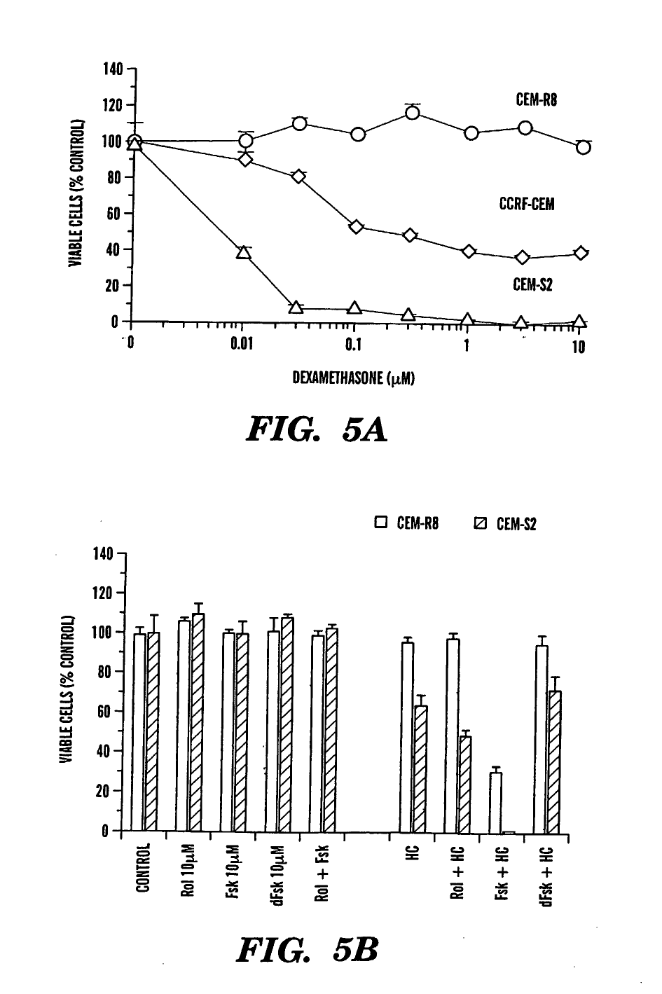

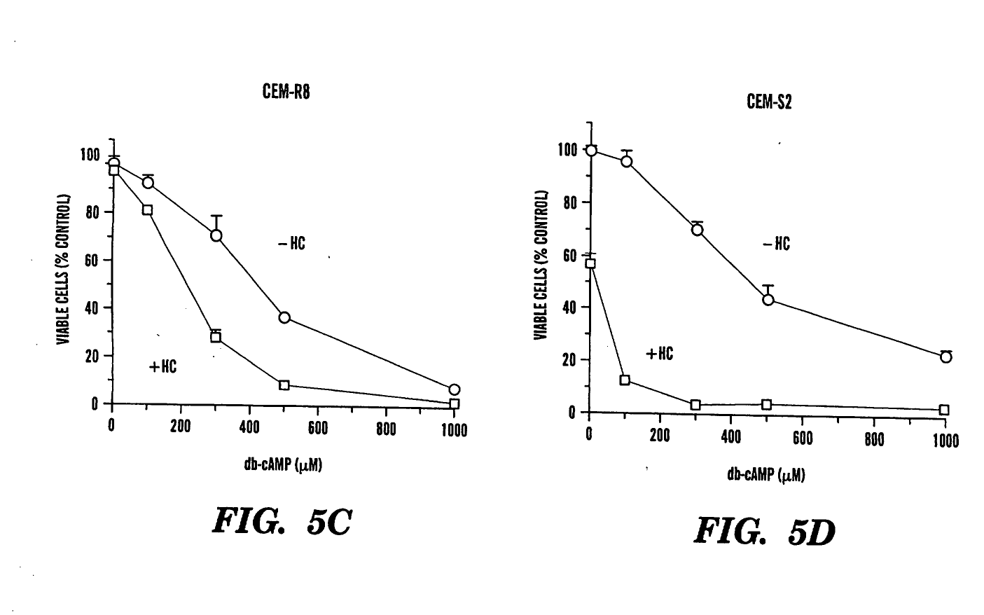

As the CCRF-CEM cell line has been utilized for seminal studies on glucocorticoid and cAMP-mediated apoptosis, we next sought to determine whether this cell line resembled B-CLL cells with respect to the ability of PDE4 inhibitors to augment glucocorticoid-mediated apoptosis and GRE transactivation^{8,11}. As prior work on PDE activity and the effect of PDE4 inhibitors in CCRF-CEM cells have utilized CEM clones of varying glucocorticoid sensitivity, we isolated both dexamethasone-sensitive and dexamethasone-resistant subclones for our studies (FIG. 5A)^{11,34}. By MTS assay, we found that, unlike B-CLL cells, 10 μM rolipram alone had no discernable effect on the viability of either dexamethasone-resistant or sensitive CCRF-CEM cells (FIG. 5B). Forskolin, either as a single agent or combined with rolipram, also had no effect on cell viability. In contrast, forskolin (10 μM), but not rolipram (10 μM), dramatically enhanced the glucocorticoid sensitivity of dexamethasone-sensitive CCRF-CEM cells, an observation that stands in striking contrast to the results previously obtained in B-CLL cells (FIG. 5B). Forskolin also induced glucocorticoid sensitivity in the glucocorticoid-resistant CCRF-CEM cell clone. When the CCRF-CEM cells were analyzed by the Hoechst 33342 apoptosis assay, comparable results were obtained (data not shown). 1,9 dideoxyforskolin, a forskolin analog that does not stimulate adenylyl cyclase, failed to augment glucocorticoid sensitivity in CCRF-CEM cells, suggesting that forskolin augments glucocorticoid-mediated CCRF-CEM apoptosis by its activity on adenylyl cyclase (FIG. 5B).

To further support the hypothesis that it is cAMP signaling and not a non-cAMP-mediated effect of forskolin that accounts for forskolin's ability to enhance glucocorticoid-mediated apoptosis in CCRF-CEM cells, we also examined the effects of combined treatment with hydrocortisone and the cAMP analog dibutyryl cAMP (dbcAMP). Apoptosis following treatment with a range of doses of dbcAMP as a single agent was similar in dexamethasone-sensitive and resistant CCRF-CEM cells, suggesting that whatever mechanism induced glucocorticoid resistance in CCRF-CEM cells did not lead to simultaneous resistance to cAMP analogs (FIG. 5C). Addition of 1 μM hydrocortisone to the same dbcAMP dose response assay resulted in marked synergy in the apoptotic effects of these two compounds for both dexamethasone-sensitive and resistant CCRF-CEM cells. These data support the hypothesis that cAMP-mediated signal transduction augments glucocorticoid-mediated apoptosis in CCRF-CEM cells regardless of the initial sensitivity of the leukemic clone to glucocorticoids.

Given the above marked discrepancy in the type of cyclic nucleotide-associated stimuli that synergize with glucocorticoids in inducing apoptosis in B-CLL and CCRF-CEM cells, we examined the effects of rolipram and forskolin on glucocorticoid-induced GRE transactivation in CCRF-CEM cells. In concurrence with the results of the apoptosis studies, we found that forskolin, but not rolipram, markedly augmented GRE transactivation in CCRF-CEM cells (FIG. 3). Forskolin as a single agent had modest effects in this assay, while rolipram as a single agent had none. The level of transactivation observed in CCRF-CEM cells was markedly higher than that observed in the B-CLL studies, most likely as a result of a higher transfection efficiency in this cell line relative to the primary leukemic cells. These studies suggest that while GR-mediated signaling is augmented by PDE4 inhibition but not adenylyl cyclase stimulation in B-CLL cells, the converse is true in the T-ALL cell line CCRF-CEM.

cAMP Levels and PDE4 Isoforms are Regulated by Rolipram and Forskolin Differently in B-CLL and CCRF-CEM Cells

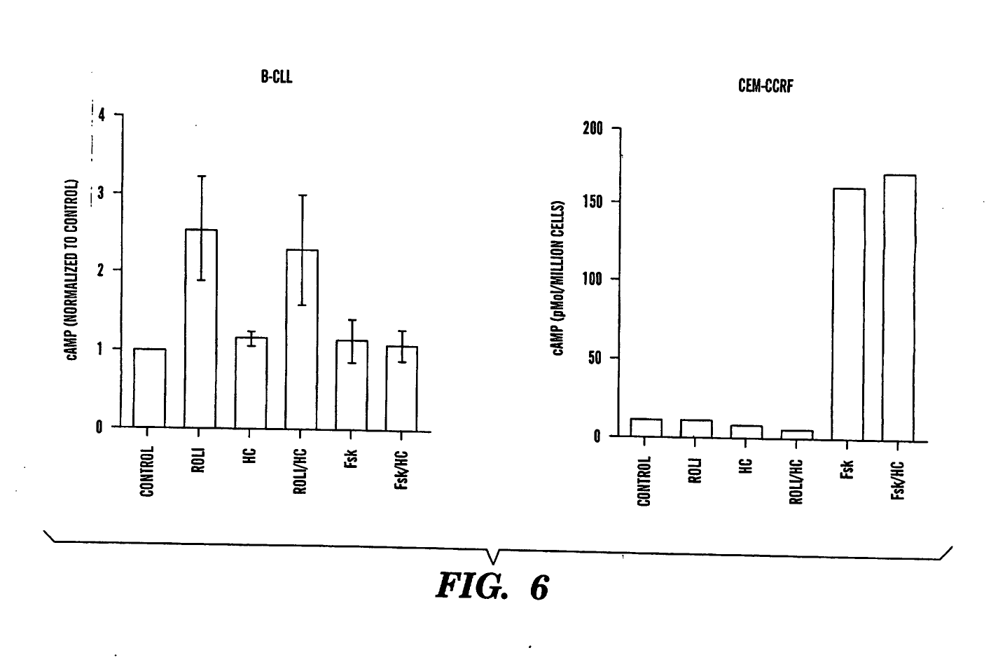

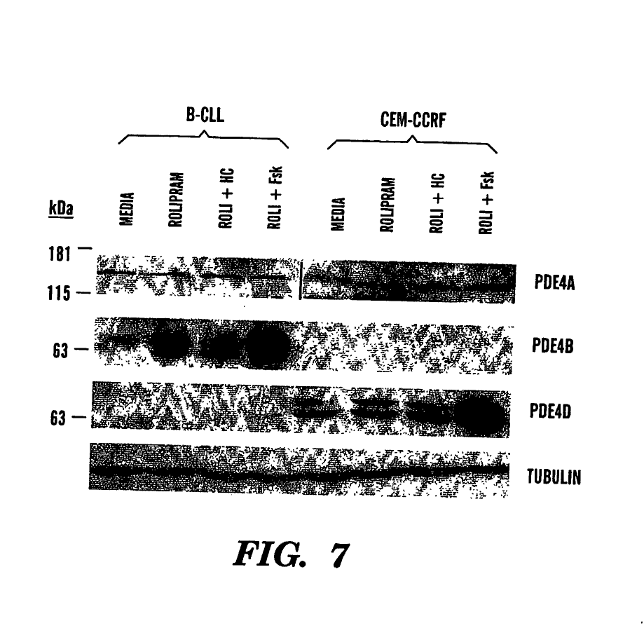

As the studies above demonstrate that B-CLL and CCRF-CEM cells differ in their response to PDE4 inhibitors and adenylyl cyclase activation, we measured cAMP levels in these two types of cells following treatment with rolipram or forskolin. As prior studies have demonstrated that glucocorticoids raise levels of cAMP in lymphocytes and potentiate the cAMP response to agents that activate adenylyl cyclase, we also examined whether glucocorticoid treatment altered the cAMP response to rolipram or forskolin³⁵⁻³⁷. In leukemic cells from six B-CLL patients examined, treatment with 10 μM rolipram for 30 minutes augmented cAMP levels 2.5±0.7-fold above that observed in untreated cells (FIG. 6)(single factor ANOVA p<0.04). In contrast, in three experiments performed, treatment of CCRF-CEM cells with rolipram had no effect while treatment with forskolin caused a marked increase in cAMP levels that was not observed in B-CLL cells (FIG. 6). Glucocorticoid treatment had no significant effect on cAMP levels in either cell type.

Given that B-CLL cells, but not CCRF-CEM cells, respond to PDE4 inhibitors with increased intracellular cAMP levels, glucocorticoid-receptor-mediated GRE activation and glucocorticoid-mediated apoptosis, we next examined the expression of PDE4 isoforms in these two cell types. Our prior studies had demonstrated that inhibition of PDE4 with rolipram resulted in marked up-regulation of PDE4B levels in B-CLL cells as judged by Western analysis³⁰. Such an observation was in keeping with studies demonstrating that pharmacologic agents result in cAMP-induced increases in levels of PDE4B and PDE4D short forms through cAMP-activated intronic enhancers³⁸. B-CLL cells were incubated with media alone, rolipram (10 μM) or rolipram combined with either hydrocortisone (1 μM) or forskolin (10 μM), followed by Western analysis for expression of PDE4A, PDE4B and PDE4D. In B-CLL cells, there was constitutive expression of a 130 kDa form of PDE4A, rolipram-inducible expression of 63 and 68 kDa forms of PDE4B and either no or very low level rolipram-induced expression of 63 and 68 kDa form of PDE4D. In contrast, in CCRF-CEM cells, we detected the same constitutive 130 kDa form of PDE4A, no PDE4B and constitutive expression of 63 and 68 kDa forms of PDE4D that were markedly increased by forskolin treatment. Thus, for at least these two cell types, rolipram-mediated inhibition of PDE4B but not PDE4D correlates with augmentation of intracellular cAMP levels, glucocorticoid-receptor-mediated GRE activation and apoptosis.

In addition to B-CLL, using methodology similar to that described above, we have also shown that PDE4 inhibitors augment sensitivity to glucocorticoids in other peripheral B-cell leukemia cells, namely, multiple myeloma cells. Combination treatment of multiple myeloma cells with PDE4 inhibitors and glucocorticoids resulted in synergistic apoptosis (data not shown). Thus, the combination therapy is useful in the treatment of peripheral B-cell neoplasm.

We have shown that treatment of B-CLL cells with PDE4 inhibitors, in the absence of exogenous stimulation of adenylyl cyclase, augments killing of these primary leukemic cells beyond that observed by adding the apoptotic effects observed with each drug class alone. Importantly, the same combined treatment does not induce apoptosis in primary human T cells. As at least two PDE4 inhibitors, roflumilast (Daxas, Altana Pharma) and cilomilast (Ariflo; GlaxoSmithKline) are in late stages of clinical development in Europe and the US, respectively, the efficacy of combined PDE4 inhibitor/glucocorticoid therapy in treatment-resistant lymphoid malignancies can be accomplished.

What factors determine whether a normal or malignant lymphoid cell will undergo apoptosis following treatment with either PDE4 inhibitors alone or in combination with glucocorticoids? In the current study, we examine two quite different examples of lymphoid cells in which activation of cAMP-mediated signaling in combination with glucocorticoid treatment induces a synergistic apoptotic effect. In B-CLL cells, the cell lineage sensitive to combined PDE4 inhibitor/glucocorticoid-induced apoptosis, treatment with rolipram alone augments cAMP levels and induces PDE4B2. In CCRF-CEM cells, the cell lineage insensitive to combined PDE4 inhibitor/glucocorticoid-induced apoptosis, treatment with rolipram neither augmented cAMP nor induced any PDE4 isoform. Notably, CCRF-CEM cells were extremely sensitive to forskolin/glucocorticoid or dbcAMP/glucocorticoid-induced apoptosis. From these results, we conclude that at least two requirements for PDE4 inhibitor-mediated lymphoid apoptosis are sufficient basal adenylyl cyclase activity to drive cAMP accumulation in a subcellular compartment (so-called “flux-mediated sensitivity”) and control of that subcellular cAMP pool by a PDE4 enzyme. Given that CCRF-CEM cells express constitutive PDE4A2, it is clearly not sufficient for a lymphoid cell to express a PDE4 isoform for it to respond to PDE4 inhibition with elevation of cAMP, compensatory up-regulation of PDE4 enzymes or apoptosis. Furthermore, given that both CCRF-CEM and B-CLL cells express PDE4A2, it seems likely that the PDE4 isoform regulating the critical subcellular pro-apoptotic cAMP compartment in B-CLL cells is PDE4B2.

However, it is also clear that adequate adenylyl cyclase activity and the presence of PDE4B2 in a lymphoid cell is not sufficient for augmentation of glucocorticoid-mediated apoptosis following treatment with PDE4 inhibitors. We have previously found that while rolipram alone does not augment cAMP levels in human whole mononuclear cells, a population consisting predominantly of T cells, combined treatment with rolipram and forskolin results in markedly higher cAMP levels than those observed with forskolin alone²¹. Despite this, in the current studies we find that the same combined treatment of purified human T cells with rolipram and forskolin does not have any significant effect on glucocorticoid-mediated apoptosis. Furthermore, multiple studies in T cells have demonstrated that they contain PDE4B enzyme and that activation or inhibition of PDE4 induces important functional changes in this cell lineage^{40 41}. Thus, in addition to adenylyl cyclase activity and a regulating PDE4 isoform, lymphoid cells must require specific “downstream” signaling targets in order for PDE4 inhibitors to activate an apoptotic cascade. While our prior work has implicated PP2A activation and BAD dephosphorylation as potentially relevant rolipram-induced events in B-CLL, a clear picture of the molecular targets that differ between these leukemic cells and peripheral T cells remain to be established²².

Why do cAMP-mediated and glucocorticoid-mediated signaling synergize in killing susceptible lymphoid populations? In one model, cAMP and glucocorticoids could activate non-interdependent signaling pathways that positively interact as a result of distinct signaling outcomes that collaborate to induce apoptosis. In another model, one signaling pathway could increase either the magnitude (quantity) or the character (quality) of signaling by the other pathway. Most, but not all, studies of the interactions between these two pathways in lymphoid cells favor the hypothesis that PKA signaling positively regulates glucocorticoid-mediated apoptotic signaling. Kiefer et al demonstrated that a GR-deficient CCRF-CEM subclone was resistant to apoptosis induced by cAMP analogs⁸. GR signaling may be not only necessary but also possibly sufficient for cAMP-mediated apoptosis in lymphoid cells, as inhibition of CRE-mediated transcriptional activation by transfection with CRE “decoy” oligonucleotides failed to protect T cell hybridomas from cAMP potentiation of GC-mediated apoptosis⁴².

We found that the best characterized GR antagonist, mefipristone (RU486), while not apoptotic in B-CLL cells when used alone, behaves as a GR agonist in the setting of co-treatment with PDE4 inhibitors or other drugs that activate PKA signaling in B-CLL cells (data not shown). Gruol et al have previously reported similar findings in immature murine T cells, where RU486 synergized with cAMP to induce apoptosis but had no activity when used alone⁴³. While we cannot therefore yet confirm that glucocorticoid-mediated signaling is required for PKA-mediated apoptosis in B-CLL, using the enantiomeric cAMP antagonist Rp-8Br-cAMPS, we do find evidence suggestive that PKA-mediated signaling is required for glucocorticoid-mediated B-CLL apoptosis. Consistent with this observation, the modest PDE4 inhibitor-induced augmentation in glucocorticoid-induced GRE transactivation we observed in transfected B-CLL cells was also inhibited by Rp-8Br-cAMPS treatment. In the more efficiently transfected CCRF-CEM cells, the augmentation in GRE transactivation following co-treatment with forskolin and glucocorticoids was far more pronounced. These data support the hypothesis that the synergistic apoptosis we observe in these two models following treatment with rolipram or forskolin combined with glucocorticoids are the result of augmented signaling through the glucocorticoid receptor. In contrast, our inability to detect augmented GRE transactivation in B-CLL cells treated with rolipram alone would seem to suggest that rolipram-induced apoptosis is independent of the GR. However, it is possible that development of a more sensitive system for assessing GR-mediated transactivation in B-CLL cells may eventually reveal effects of PDE4 inhibitor on the basal activity of this signaling pathway.

If the glucocorticoid receptor is the relevant target of PKA in combined PDE4 inhibitor/glucocorticoid therapy, what does PKA phosphorylate and how does the resulting transcriptional complex trigger apoptosis? Although PKA has been reported to be associated with the GR and to modulate GR-mediated transcriptional activity, consistent evidence of functionally relevant phosphorylation of the GR itself by PKA is lacking^{44 13}. Alternatively, PKA may phosphorylate other transcription factors or co-activators or co-repressors with which the GR interacts^{13 45}. Studies of glucocorticoid-induced lymphoid apoptosis have come to differing conclusions with regard to the role of GR-mediated transactivation through GREs or transrepression of NFκB or AP1⁶. A murine “knock-in” for the A458T dimerization-defective GR that cannot activate GRE-containing promoters demonstrated that glucocorticoids no longer induce apoptosis in the thymocytes of such mice, while glucocorticoid-mediated AP-1 and NFκB transrepression remains intact⁴⁶. If GR-mediated apoptosis does indeed occur through transactivation, a potentially relevant target is the pro-apoptotic BH3-only Bc12 family member Bim. Bim is up-regulated by both cAMP-mediated and glucocorticoid-mediated signaling, although it is not clear whether cAMP-mediated up-regulation of Bim is dependent upon functional GR, nor whether glucocorticoid-mediated up-regulation of Bim requires PKA function^{47,48}. In contrast, glucocorticoid-mediated killing of Jurkat cells has been argued to be independent of GR-mediated transcriptional activation⁴⁹. A recent study of CCRF-CEM cells transfected with two GR mutants that lack, respectively, the ability to transactivate GRE promoters or transrepress NFκB suggested that either GR activity is sufficient for glucocorticoid-mediated apoptosis in these lymphoid cells⁵⁰. A readily transfectable cell line in which PDE4 inhibition induces apoptosis will be required in order to examine more critically whether PDE4 inhibitor-associated synergy with GR signaling proceeds through a transactivation or transrepression pathway.

Recent studies by other groups have supported the concept that PDE4 may prove to be an important therapeutic target in resistant human lymphoid malignancies. A chip analysis by Shipp et al demonstrated that mRNA expression of one PDE4 gene family member, PDE4B, correlates with resistance of diffuse large B cell lymphomas (DLBCL) to standard CHOP chemotherapy⁵¹. Further, the authors found that the data set of Alizadeh et al, a completely independent study of DLBCL, corroborated the finding of a negative prognostic influence of high level expression of PDE4B transcripts⁵². Given our finding that inhibition of PKA in B-CLL cells with Rp-Br-cAMPS leads to striking resistance to hydrocortisone-induced apoptosis, we hypothesize that high level expression of PDE4B reduces PKA activation in DLBCL cells, thereby rendering them less sensitive to the apoptotic effects of prednisone, a component of the CHOP regimen. Given the expression of PDE4B2 in rolipram-sensitive B-CLL but not rolipram-insensitive CCRF-CEM cells, it will be of interest to determine whether isoform-specific subcellular targeting of PDE4B2 accounts for its ability to regulate a cAMP pool that is capable of initiating an apoptotic cascade in susceptible lymphoid populations. Regardless of the mechanism by which PDE4 inhibitor/glucocorticoid synergy occurs, the coming availability of PDE4 inhibitors in the clinic should eventually allow us to test the concept that this class of drugs may aid in reversing resistance to glucocorticoid-containing chemotherapy regimens.

TABLE 1

| Patient | Stage | Prior therapy^{a} | WBC | No Rx | Roli^{a} | HC^{b} | R/HC | Additive vs Obs. |
|---|---|---|---|---|---|---|---|---|
| #1 | Stage 1 | No Rx | 13 | 10 | 17 | 19 | 27 | 16 vs 17 |
| #2 | Stage 3 | C, F, R, Cy, St | 295 | 11 | 13 | 16 | 65 | 7 vs 54 |
| #3 | Stage 4 | CHOP, F | 87 | 13 | 24 | 41 | 67 | 39 vs 54 |
| #4 | Stage 3 | F | 228 | 23 | 34 | 42 | 63 | 30 vs 40 |
| #5 | Stage 3 | C, F, R, St | 66 | 25 | 37 | 43 | 66 | 30 vs 41 |
| #6 | Stage 1 | No Rx | 30 | 27 | 45 | 33 | 60 | 24 vs 33 |
| #7 | Stage 1 | No Rx | 15 | 31 | 67 | 47 | 69 | 52 vs 38 |
| #8 | Stage 2 | No Rx | 51 | 33 | 62 | 60 | 76 | 56 vs 43 |
| #9 | Stage 4 | No Rx | 147 | 35 | 81 | 71 | 85 | 77 vs 44 |
| #10 | Stage 2 | No Rx | 57 | 44 | 58 | 75 | 78 | 45 vs 34 |
| #11 | Stage 3 | C, St | 12 | 61 | 75 | 73 | 76 | 27 vs 15 |

^{a}Chemotherapy: Chlorambucil (C), fludarabine (F), rituxan (R), cyclophosphamide (Cy), prednisone (St), combination chemotherapy with cyclophosphamide, doxorubicin, vincristine and prednisone (CHOP).
^{a}Rolipram was used at 10 μM.
^{b}Hydrocortisone was used at 1 μM.

### REFERENCES

1. McConkey D J, Orrenius S, Jondal M. Agents that elevate cAMP stimulate DNA fragmentation in thymocytes. J. Immunol. 1990; 145:1227-1230
2. Kizaki H, Suzuki K, Tadakuma T, Ishimura Y. Adenosine receptor-mediated accumulation of cAMP-induced T-lymphocyte death through internucleosomal DNA cleavage. J. Biol. Chem. 1990; 265:5280-5284
3. Lomo J, Blomhoff H K, Beiske K, Stokke T, Smeland E B. TGF-B1 and cyclic AMP promote apoptosis in resting human B lymphocytes. J. Immunol. 1995; 154:1634-1643
4. Wyllie A H, Kerr, J F R, Currie, A R. Cell death: The significance of apoptosis. Int. Rev. Cytology. 1980; 68:251-306
5. Cohen J J, Duke R C. Glucocorticoid activation of a calcium dependent endonuclease in thymocyte nuclei leads to cell death. J Immunol. 1984; 132:38-42
6. Greenstein S, Ghias K, Krett N L, Rosen S T. Mechanisms of glucocorticoid-mediated apoptosis in hematological malignancies. Clin Cancer Res. 2002; 8:1681-1694
7. Harrigan M T, Baugman G, Campbell N F, Bourgeois S. Isolation and characterization of glucocorticoid and cylic-AMP-induced genes in T lymphocytes. Mol. Cell Biol. 1989; 9:3438-3446
8. Kiefer J, Okret S, Jondal M, McConkey D J. Functional glucocorticoid receptor expression is required for cAMP-mediated apoptosis in a human leukemic T cell line. J. Immunol. 1995; 155:4525-4528
9. Dowd D R, Miesfield R L. Evidence that glucocorticoid and cyclic AMP-induced apoptotic pathways in lymphocytes share distal events. Mol Cell Biol. 1992; 12:3600-3608
10. Medh R D, Saeed M F, Johnson B H, Thompson E B. Resistance of human leukemic CEM-C1 cells is overcome by synergism between glucocorticoid and protein kinase A pathways: correlation with myc suppression. Cancer Research. 1998; 58:3684-3693
11. Ogawa R, Streiff M B, Bugayenko A, Kato G J. Inhibition of PDE4 phosphodiesterase activity induces growth suppression, apoptosis, glucocorticoid sensitivity, p53, and p21 WAF1/CIP1 proteins in human acute lymphoblastic leukemia cells. Blood. 2002; 99:3390-3397
12. Refojo D, Liberman A C, Giacomini D, Carbia Nagashima A, Graciarena M, Echenique C, Paez Pereda M, Stalla G, Holsboer F, Arzt E. Integrating systemic information at the molecular level: cross-talk between steroid receptors and cytokine signaling on different target cells. Ann N Y Acad Sci. 2003; 992:196-204
13. Doucas V, Shi Y, Miyamoto S, West A, Verma I, Evans R M. Cytoplasmic catalytic subunit of protein kinase A mediates cross-repression by NF-kB and the glucocorticoid receptor. Proc Natl Acad Sci USA. 2000; 97:11893-11898
14. Heck S, Bender K, Kullmann M, Gottlicher M, Herrlich P, Cato A C. I kappaB alpha-independent downregulation of NF-kappaB activity by glucocorticoid receptor. Embo J. 1997; 16:4698-4707
15. Nissen R M, Yamamoto K R. The glucocorticoid receptor inhibits NFkappaB by interfering with serine-2 phosphorylation of the RNA polymerase II carboxy-terminal domain. Genes Dev. 2000; 14:2314-2329
16. De Bosscher K, Vanden Berghe W, Haegeman G. The interplay between the glucocorticoid receptor and nuclear factor-kappaB or activator protein-1: molecular mechanisms for gene repression. Endocr Rev. 2003; 24:488-522
17. Reichardt H M, Tuckermann J P, Gottlicher M, Vujic M, Weih F, Angel P, Herrlich P, Schutz G. Repression of inflammatory responses in the absence of DNA binding by the glucocorticoid receptor. Embo J. 2001; 20:7168-7173
18. Epstein P M, Hachisu R. Cyclic nucleotide phosphodiesterase in normal and leukemic human lymphocytes and lymphoblasts. Adv. Cyclic Nucleotide Protein Phosphorylation Res. 1984; 16:303-324
19. Lerner A, Kim B, Lee R. The cAMP signaling pathway as a therapeutic target in lymphoid malignancies. Leuk & Lymph. 2000; 37:39-51
20. Jiang X, Paskind M, Weltzien R, Epstein P M. Expression and regulation of mRNA for distinct isoforms of cAMP-specific phosphodiesterase (PDE4) in mitogen-stimulated and leukemic human lymphocytes. Cell Biochem. Biophys. 1998; 28:135-160
21. Kim D H, Lerner A. Type 4 cyclic adenosine monophosphate phosphodiesterase as a therapeutic target in chronic lymphocytic leukemia. Blood. 1998; 92:2484-2494
22. Moon E-Y, Lerner A. PDE4 inhibitors activate a mitochondrial apoptotic pathway in chronic lymphocytic leukemia cells that is regulated by protein phosphatase 2A. Blood. 2003; 101:4122-4130
23. Tiwari S, Felekkis K, Moon E, Flies A, Sherr D, Lerner A. Among circulating hematopoietic cells, B-CLL uniquely expresses functional EPAC1 but EPAC1-mediated Rap1 activation does not account for PDE4 inhibitor-induced apoptosis. Blood. 2004; 103:2661-2667
24. Giembycz M. Phosphodiesterase 4 inhibitors and the treatment of asthma: where are we now and where do we go from here? Drugs. 2000; 59:193-212
25. Bundschuh D, Eltze M, Barsig J, Wollin L, Hatzelmann A, Beume R. In vivo efficacy in airway models of roflumilast, a novel orally active PDE4 inhibitor. J. Pharmacol. Exp. Ther. 2001; 297:280
26. Lee R, Wolda S, Moon E, Esselstyn J, Hertel C, Lerner A. PDE7A is expressed in human B lymphocytes and is up-regulated by elevation of intracellular cAMP. Cellular Signalling. 2002; 14:277-284
27. Foley G E, Lazarus H, Farber S, Uzman B G, Boone B A, McCarthy R E. Continuous Culture of Human Lymphoblasts from Peripheral Blood of a Child with Acute Leukemia. Cancer. 1965; 18:522-529
28. Tiwari S, Felekkis K, Moon E, Flies A, Sherr D, Lerner A. Among circulating hematopoietic cells, B-CLL uniquely expresses functional EPAC1 but EPAC1-mediated Rap1 activation does not account for PDE4 inhibitor-induced apoptosis. Blood. 2003; (in press).
29. Umesono K, Evans R M. Determinants of target gene specificity for steroid/thyroid hormone receptors. Cell. 1989; 57:1139-1146
30. Moon E, Lee R, Near R, Weintraub L, Wolda S, Lerner A. Inhibition of PDE3B augments PDE4-inhibitor-induced apoptosis in leukemic cells from a subset of patients with chronic lymphocytic leukemia. Clin. Cancer Res. 2002; 8:589-595
31. Bolger G B, Erdogan S, Jones R E, Loughney K, Scotland G, Hoffmann R, Wilkinson I, Farrell C, Houslay M D. Characterization of five different proteins produced by alternatively spliced mRNAs from the human cAMP-specific phosphodiesterase PDE4D gene. Biochem J. 1997; 328:539-548
32. Miller A, Vogt G, Pearce B. The phosphodiesterase type 4 inhibitor, rolipram, enhances glucocorticoid receptor function. Neuropsychopharmacol. 2002; 27:939-948
33. Klussmann E, Maric K, Wiesner B, Beyermann M, Rosenthal W. Protein kinase A anchoring proteins are required for vasopressin-mediated translocation of aquaporin-2 into cell membranes of renal principal cells. J Biol Chem. 1999; 274:4934-4938
34. Epstein P M, Mills J S, Ross C P, Strada S J, Hersh E M, Thompson W J. Increased cyclic nucleotide phosphodiesterase activity associated with proliferation and cancer in human and murine lymphoid cells. Cancer Res. 1977; 37:4016-4023
35. Marone G, Lichtenstein L M, Plaut M. Hydrocortisone and human lymphocytes: increases in cyclic adenosine 3′:5′-monophosphate and potentiation of adenylate cyclase-activating agents. J Pharmacol Exp Ther. 1980; 215:469-478
36. Michel M C, Knapp J, Ratjen H. Sensitization by dexamethasone of lymphocyte cyclic AMP formation: evidence for increased function of the adenyl cyclase catalyst. Brit. J. Pharmacol. 1994; 113:240-246
37. Baus E, Van Laethem F, Andris F, Rolin S, Urbain J, Leo O. Dexamethasone increases intracellular cyclic AMP concentration in murine T lymphocyte cell lines. Steroids. 2001; 66:39-47
38. Vicini E, Conti M. Characterization of an intronic promoter of a cyclic adenosine 3′,5′-monophosphate (cAMP)-specific phosphodiesterase gene that confers hormone and cAMP inducibility. Mol Endocrinol. 1997; 11:839-850
39. McConkey D J, Orrenius S, Okret S, Jondal M. Cyclic AMP potentiates glucocorticoid-induced endogenous endonuclease activation in thymocytes. Faseb J. 1993; 7:580-585
40. Baroja M L, Cieslinski L B, Torphy T J, Wange R L, Madrenas J. Specific CD3 epsilon association of a phosphodiesterase 4B isoform determines its selective tyrosine phosphorylation after CD3 ligation. J Immunol. 1999; 162:2016-2023
41. Arp J, Kirchhof M G, Baroja M L, Nazarian S H, Chau T A, Strathdee C A, Ball E H, Madrenas J. Regulation of T-cell activation by phosphodiesterase 4B2 requires its dynamic redistribution during immunological synapse formation. Mol Cell Biol. 2003; 23:8042-8057
42. Muller Igaz L, Refojo D, Costas M A, Holsboer F, Arzt E. CRE-mediated transcriptional activation is involved in cAMP protection of T-cell receptor-induced apoptosis but not in cAMP potentiation of glucocorticoid-mediated programmed cell death. Biochim Biophys Acta. 2002; 1542:139-148
43. Groul D J, Altschmied J. Synergistic induction of apoptosis with glucocorticoids and 3′-5′-cyclic adenosine monophosphate reveals agonist activity by RU486. Molec Endocrin. 1993; 7:104-113
44. Rangarajan P, Umesono K, Evans R. Modulation of glucocorticoid receptor by protein kinase A. Mol Endocrin. 1992; 6:1451-1457
45. Kurihara I, Shibata H, Suzuki T, Ando T, Kobayashi S, Hayashi M, Saito I, Saruta T. Expression and regulation of nuclear receptor coactivators in glucocorticoid action. Mol Endocrinol. 2002; 189:181-189
46. Reichardt H M, Kaestner K H, Tuckermann J, Kretz O, Wessely O, Bock R, Gass P, Schmid W, Herrlich P, Angel P, Schutz G. DNA binding of the glucocorticoid receptor is not essential for survival. Cell. 1998; 93:531-541
47. Wang Z, Malone M H, He H, McColl K S, Distelhorst C W. Microarray analysis uncovers the induction of the proapoptotic BH3-only protein Bim in multiple models of glucocorticoid-induced apoptosis. J Biol Chem. 2003; 278:23861-23867
48. Zhang L, Insel P A. The pro-apoptotic protein Bim: a convergence point for cAMP/PKA- and glucocorticoid-promoted apoptosis of lymphoid cells. J Biol Chem. 2004
49. Helmburg A, Auphan N, Caelles C, Karin M. Glucocorticoid-induced apoptosis of human leukemic cells is caused by the repressive function of the glucocorticoid receptor. EMBO J. 1995; 14:452-460
50. Tao Y, Williams-Skipp C, Scheinman R I. Mapping of glucocorticoid receptor DNA binding domain surfaces contributing to transrepression of NF-kappa B and induction of apoptosis. J Biol Chem. 2001; 276:2329-2332
51. Shipp M A, Ross K N, Tamayo P, Weng A P, Kutok J L, Aguiar R C, Gaasenbeek M, Angelo M, Reich M, Pinkus G S, Ray T S, Koval M A, Last K W, Norton A, Lister T A, Mesirov J, Neuberg D S, Lander E S, Aster J C, Golub T R. Diffuse large B-cell lymphoma outcome prediction by gene-expression profiling and supervised machine learning. Nat Med. 2002; 8:68-74
52. Alizadeh A A, Eisen M B, Davis R E, Ma C, Lossos I S, Rosenwald A, Boldrick J C, Sabet H, Tran T, Yu X, Powell J I, Yang L, Marti G E, Moore T, Hudson J, Jr., Lu L, Lewis D B, Tibshirani R, Sherlock G, Chan W C, Greiner T C, Weisenburger D D, Armitage J O, Warnke R, Levy R, Wilson W, Grever M R, Byrd J C, Botstein D, Brown P O, Staudt L M. Distinct types of diffuse large B-cell lymphoma identified by gene expression profiling. Nature. 2000; 403:503-511

All references described herein are incorporated by reference in their entirety.

What is claimed:

1. A method for treating an individual having a peripheral B-cell neoplasm, comprising:
a. selecting an individual having symptoms of peripheral B-cell neoplasm; and
b. administering to said individual a therapeutically effective amount of i) an inhibitor that specifically inhibits Type 4 cyclic adenosine monophosphate phosphodiesterases (a PDE4 inhibitor); and ii) a glucocorticoid to interact synergistically to treat said individual.

2. The method of claim 1, wherein the peripheral B-cell neoplasm is selected from the group consisting of B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue (MALT type), nodal marginal zone lymphoma, splenic marginal zone lymphoma, hairy cell leukemia, plasmacytoma, diffuse large B-cell lymphoma, Burkitt lymphoma, multiple myeloma, B-cell non-Hodgkin's lymphoma and Waldenstrom's macroglobulineamia.

3. The method of claim 1, wherein the peripheral B-cell neoplasm is chronic lymphocytic leukemia.

4. The method of claim 1 wherein the inhibitor is selected from the group consisting of rolipram, RO20-1724, piclamilast, NCS-613, D-4418, mesopram, CI-1018, a benzodioxole derivative, PMNPQ (6-(4-pyridylmethyl)-8-(3-nitrophenyl)quinoline, roflumilast, a pthalazinone, T-440, cis 4-cyano-4-(3-cyclopentyloxy-4-met-hoxyphenyl)cyclohexan-1-carboxylic acid, 2-carbomethoxy-4-cyano-4-(3-cyclo-propylmethoxy-4-difluoromethoxyphenyl)cyclohexan-1-one; cis-[4-cyano-4-(3-cyclopropylmethoxy-4-difluoromethoxyphenyl)cyclohexan-1-ol], cilomalast, L-826,141 [4-{2-(3,4-Bisdifluromethoxyphenyl)-2-{4-(1,1,1,3,3,3-hexafluoro-2-hydroxypropan-2-yl)-phenyl]-ethyl}-3-methylpyridine-1-oxide], AWD 12-343; 7-benzylamino-6-chloro-2-piperazino-pteridine, AWD-12-281, arofylline, and pharmaceutically acceptable salts, esters, pro-drugs, and analogues thereof.

5. The method of claim 4, wherein the inhibitor is roflumilast or cilomalast.

6. The method of claim 4, wherein the inhibitor is rolipram or RO20-1724.

7. The method of claim 1, wherein the glucocorticoid is selected from the group consisting of betamethasone, budesonide, cortisone, dexamethasone, hydrocortisone, methylprednisolone, prednisolone, prednisone, and triamcinolone.

8. The method of claim 1, wherein said patient is unresponsive to chemotherapy with alkylating agents.

9. The method of claim 1, further comprising administering an alkylating agent.

10. The method of claim 9, wherein the alkylating agent is selected from the group consisting of chlorambucil, adenosine analogs, fludarabine, carboplatin and paclitaxel.

11.-13. (canceled)

14. A kit for use in the treatment of peripheral B-cell neoplasm comprising a carrier containing one or more components, wherein a first component comprises an inhibitor that specifically inhibits Type 4 cyclic adenosine monophosphate phosphodiesterases (a PDE4 inhibitor) and a second component comprises a glucocorticoid.

15. A kit for synergistically causing apoptosis of peripheral B-cell neoplastic cells in an individual with peripheral B-cell neoplasm comprising: a. an inhibitor that specifically inhibits Type 4 cyclic adenosine monophosphate phosphodiesterases (a PDE4 inhibitor) or a pharmaceutically acceptable salt thereof and a pharmaceutically acceptable carrier or diluent in a first dosage form; b. an amount of a glucocorticoid or a pharmaceutically acceptable salt thereof and a pharmaceutically acceptable carrier or diluent in a second unit dosage form; c. a container for containing said first and second dosage form; and d. directions for the administration to an individual.

16. The method of claim 4, wherein the glucocorticoid is selected from the group consisting of betamethasone, budesonide, cortisone, dexamethasone, hydrocortisone, methylprednisolone, prednisolone, prednisone, and triamcinolone.
